(19) United States
(12) Patent Application Publication
(10) Pub. No.: US 2023/0405566 A1
(43) Pub. Date: Dec. 21, 2023

(54) SYNTHESIS AND ORGANIZATION OF GOLD-PEPTIDE NANOPARTICLES FOR CATALYTIC ACTIVITIES

(71) Applicant: KING ABDULLAH UNIVERSITY OF SCIENCE AND TECHNOLOGY, Thuwal (SA)

(72) Inventors: Charlotte A.E. HAUSER, Thuwal (SA); Hepi Hari SUSAPTO, Thuwal (SA)

(21) Appl. No.: 18/218,162

(22) Filed: Jul. 5, 2023

Related U.S. Application Data
(63) Continuation-in-part of application No. 17/401,542, filed on Aug. 13, 2021.
(60) Provisional application No. 63/067,962, filed on Aug. 20, 2020.
(60) Provisional application No. 63/067,913, filed on Aug. 20, 2020.
(60) Provisional application No. 63/358,563, filed on Jul. 6, 2022.
(60) Provisional application No. 63/523,658, filed on Jun. 28, 2023.

(51) Int. Cl. B01J 31/18

(52) U.S. Cl. CPC B01J 31/1805; B01J 2231/64

(57) ABSTRACT

A facile strategy is used to synthesize the gold nanoparticles via a green and simple approach showing self-alignment on the assembled nanofibers of ultrashort oligopeptides as a composite material. A photochemical reduction method is used, without requiring any external chemical reagents for the reduction of gold ions and producing gold nanoparticles of size ca. 5 nm under mild UV light exposure. The specific arrangement of gold nanoparticles on peptide nanofibers may indicate electrostatic interactions of two components and interactions with the amino group of the peptide building block. The gold-peptide nanoparticle composites show the ability as a catalyst to degradation of environmental pollutant p-nitrophenol to p-aminophenol, and the reaction rate constant for catalysis is 0.057 min⁻¹ at a 50-fold dilute sample of 2 mg/mL and 0.72 mM gold concentration in the composites. This colloidal strategy helps researchers to fabricate the metalized bioorganic composites for biomedical and bio-catalysis applications.

### CROSS-REFERENCE TO RELATED APPLICATIONS

This application claims benefit of priority of U.S. patent application Ser. No. 17/401,542 entitled, “SCAFFOLDS FROM SELF-ASSEMBLING TETRAPEPTIDES SUPPORT 3D SPREADING, OSTEOGENIC DIFFERENTIATION AND ANGIOGENESIS OF MESENCHYMAL STEM CELLS” filed Aug. 13, 2021, which in turn claims priority to U.S. Provisional Patent Application No. 63/067,913, entitled “PEPTIDE COMPOUND WITH REPETITIVE SEQUENCE” filed Aug. 20, 2020 and to U.S. Provisional Patent Application No. 63/067,962, entitled “TETRAMERIC SELF-ASSEMBLING PEPTIDES SUPPORT 3D SPREADING AND OSTEOGENIC DIFFERENTIATION OF MESENCHYMAL STEM CELLS” filed Aug. 20, 2020, of which the present application is a continuation-in-part application. This application also claims priority to U.S. Provisional Patent Application No. 63/358,563, entitled “SELF-ASSEMBLING PEPTIDES FOR DRUG DELIVERY APPLICATIONS” filed Jul. 6, 2022 and to U.S. Provisional Patent Application No. 63/525,658, entitled “DESIGN AND DEVELOPMENT OF PEPTIDE-MODIFIED ANTINEOPLASTIC DRUGS FOR TARGETING BREAST CANCER CELLS” filed Jun. 28, 2023. The entire contents and disclosures of these patent applications are incorporated herein by reference in their entirety.

This application refers to “SYNTHESIS AND ORGANIZATION OF GOLD-PEPTIDE NANOPARTICLES FOR CATALYTIC ACTIVITIES,” in American Chemical Society journal published on Jan. 6, 2022. The entire contents and disclosures of these patent applications are incorporated herein by reference.

### BACKGROUND

### Field of the Invention

The present disclosure relates generally to synthesis and organization of gold-peptide nanoparticles for catalytic activities.

### Background of the Invention

A significant development in the synthesis strategies of metal-peptide composites and their applications in biomedical and bio-catalysis has been reported. However, the random aggregation of gold nanoparticles provides the opportunity to find alternative fabrication strategies of gold-peptide composite nanomaterials.

### SUMMARY

According to first broad aspect, the present disclosure provides a metal-peptide nanoparticle comprising at least one metal nanoparticle; and at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3, wherein the metal-peptide nanoparticle is distributed on the peptide.

According to a second broad aspect, the present disclosure provides a biocatalyst comprising at least one metal nanoparticle; and at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA, wherein X is comprised of a polar amino acid; and wherein n being an integer being selected from 0-5 and m being an integer being selected from 0-3, wherein the metal nanoparticle is distributed on the peptide.

According to a third broad aspect, the present disclosure provides a kit comprising at least one metal nanoparticle; and at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3, wherein the metal nanoparticle is distributed on the peptide.

According to a fourth broad aspect, the present disclosure provides a device for applying a metal-peptide nanoparticle, wherein the metal-peptide nanoparticle comprises at least one metal nanoparticle; and at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3, wherein the metal-peptide nanoparticle is distributed on the peptide.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

The accompanying drawings, which are incorporated herein and constitute part of this specification, illustrate exemplary embodiments of the invention, and, together with the general description given above and the detailed description given below, serve to explain the features of the invention.

FIG. 1. illustrates UV-VIS spectra and pictures after UV light exposure at different gold concentrations, according to one embodiment of the present disclosure. (FIG. 1A graphically illustrates absorption spectra of GPNP composite suspension composed of 1.83 mM IVFK peptide and gold salt at various concentrations of 0.18, 0.36, and 0.72 mM. FIG. 1B illustrates a pictorial representation of color changing of the same concentrations of GPNPs used in FIG. 1A after UV irradiation at 254 nm wavelength.)

FIG. 2 illustrates a morphology characterization of GPNP composites, according to one embodiment of the present disclosure.

FIG. 3 illustrates a crystallinity investigation by HR-TEM and XRD, according to one embodiment of the present disclosure.

FIG. 4 graphically illustrates FTIR and XPS for binding studies, according to one embodiment of the present disclosure. (FIG. 4A illustrates a Fourier transmission infrared FTIR spectra and high-resolution XPS and deconvolution of Au4f@GPNP (FIG. 4B), N1s@IVFK (FIG. 4C), and N1s@GPNP (FIG. 4D).)

FIG. 5 illustrates catalytic activity of GPNPs for the reduction of p-nitrophenol, according to one embodiment of the present disclosure. (FIG. 5A illustrates a chemical structure of p-nitrophenol and p-aminophenol. FIG. 5B graphically illustrates a catalytic reduction of p-nitrophenol into p-aminophenol in the presence of 2 mg/mL peptides and 0.72 mM GPNP composites (50x diluted). FIG. 5C graphically illustrates Absorbance of p-nitrophenol at 400 nm as a function of time. FIG. 5D graphically illustrates a reaction rate constant for catalysis.)

FIG. 6 illustrates GPNP stability over 14 days, according to one embodiment of the present disclosure.

FIG. 7 illustrates photochemical reduction of HAuCl₄ in the absence of IVFK peptide, according to one embodiment of the present disclosure. (FIG. 7A graphically illustrates UV-Vis absorption spectra of 0.72 mM gold chloride before and after 30 min of UV irradiation without addition of peptide. FIG. 7B shows a pictorial representation of 0.72 mM HAuCl₄ after UV irradiation in the absence of IVFK peptide.)

FIG. 8 graphically illustrates Zeta potential of GPNPs, according to one embodiment of the present disclosure.

FIG. 9 illustrates an XPS survey spectra of GPNPs, according to one embodiment of the present disclosure.

FIG. 10 illustrates catalytic activity of IVFK peptide for the reduction of p-nitrophenol, according to one embodiment of the present disclosure.

FIG. 11 illustrates a catalytic conversion of 4-nitrophenol at high concentration of gold-peptide nanoparticle composites (2 mg/mL IVFK and 0.72 mM gold concentration), according to one embodiment of the present disclosure.

FIG. 12 illustrates a chemical Structure of a Tetramer Peptide and fabrication of GPNP composites with UV (254 nm) Light, according to one embodiment of the present disclosure.

FIG. 13 illustrates a dropper/closure device for applying a metal-peptide nanoparticle, according to one embodiment of the present disclosure.

FIG. 14 illustrates a squeeze bottle pump spray device for applying a metal-peptide nanoparticle, according to one embodiment of the present disclosure.

FIG. 15 illustrates an airless and preservative-free spray device for applying a metal-peptide nanoparticle, according to one embodiment of the present disclosure.

FIG. 16 illustrates an injectable device for applying a metal-peptide nanoparticle, according to one embodiment of the present disclosure.

### DETAILED DESCRIPTION OF THE INVENTION

### Definitions

Where the definition of terms departs from the commonly used meaning of the term, applicant intends to utilize the definitions provided below, unless specifically indicated.

Unless defined otherwise, all technical and scientific terms used herein have the same meaning as is commonly understood to which the claimed subject matter belongs. In the event that there is a plurality of definitions for terms herein, those in this section prevail. All patents, patent applications, publications and published nucleotide and amino acid sequences (e.g., sequences available in GenBank or other databases) referred to herein are incorporated by reference. Where reference is made to a URL or other such identifier or address, it is understood that such identifiers can change and information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

For purposes of the present invention, a value or property is “based” on a particular value, property, the satisfaction of a condition, or other factor, if that value is derived by performing a mathematical calculation or logical decision using that value, property or other factor.

For purposes of the present invention, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. For purposes of the present invention, directional terms such as “top,” “bottom,” “upper,” “lower,” “above,” “below,” “left,” “right,” “horizontal,” “vertical,” “up,” “down,” etc., are used merely for convenience in describing the various embodiments of the present disclosure. The embodiments of the present disclosure may be oriented in various ways. For example, the diagrams, apparatuses, etc., shown in the drawing figures may be flipped over, rotated by 90° in any direction, reversed, etc.

For purposes of the present invention, the term “comprising”, the term “having”, the term “including,” and variations of these words are intended to be open-ended and mean that there may be additional elements other than the listed elements.

For purposes of the present invention, the term “amino acid” refers to the molecules composed of terminal amine and carboxylic acid functional groups with a carbon atom between the terminal amine and carboxylic acid functional groups sometimes containing a side chain functional group attached to the carbon atom (e.g. a methoxy functional group, which forms the amino acid serine). Typically, amino acids are classified as natural and non-natural. Examples of natural amino acids include glycine, alanine, valine, leucine, isoleucine, proline, phenylananine, tyrosine, tryptophan, serine, threonine, cysteine, methionine, asparagine, glutamine, lysine, arginine, histidine, aspartate, and glutamate, among others. Examples of non-natural amino acids include L-3,4-dihydroxyphenylalanine, 2-aminobutyric acid, dehydralanine, g-carboxyglutamic acid, carnitine, gamma-aminobutyric acid, hydroxyproline, and selenomethionine, among others. In the context of this invention, it should be appreciated that the amino acids may be the L-optical isomer or the D-optical isomer.

For purposes of the present invention, the term “biomolecule” refers to the conventional meaning of the term biomolecule, i.e., a molecule produced by or found in living cells, e.g., a protein, a carbohydrate, a lipid, a phospholipid, a nucleic acid, etc.

For purposes of the present invention, the term “carrier” refers to relatively nontoxic chemical compounds or agents that facilitate the incorporation of a drug into cells or tissues.

For purposes of the present invention, the term “cell-laden tissue scaffold” refers to the addition of cells on scaffold to form a tissue.

For purposes of the present invention, the term “effective amount” or “effective dose” or grammatical variations thereof refers to an amount of an agent sufficient to produce one or more desired effects. The effective amount may be determined by a person skilled in the art using the guidance provided herein.

For purposes of the present invention, the term “gel” and “hydrogel” are used interchangeably. These terms refer to a network of polymer chains, entrapping water or other aqueous solutions, such as physiological buffers, of over 99% by weight.

For purposes of the present invention, the term “microstructure” refers to a structure having at least one dimension smaller than 1 mm. A nanostructure is one type of microstructure.

For purposes of the present invention, the term “nanostructure” refers to a structure having at least one dimension on the nanoscale, i.e., a dimension between 0.1 and 100 nm.

For purposes of the present invention, the term “patient” and the term “subject” refer to an animal, which is the object of treatment, observation or experiment. By way of example only, a subject may be, but is not limited to, a mammal including, but not limited to, a human.

For purposes of the present invention, the term “room temperature” or “ambient temperature” refers to a temperature of from about 20° C. to about 25° C.

For purposes of the present invention, the term “scaffolds” as used herein means the ultra-short peptide or other polymer materials in the bioinks that provide support for the cellular components.

For purposes of the present invention, the term “seeding” refers to a method to add cells on a scaffold to produce surfaces.

For purposes of the present invention, the term “subject” and the term “patient” refers to an entity which is the object of treatment, observation, or experiment. By way of example only, a “subject” or “patient” may be, but is not limited to a human, a mammal, a reptile, a bird, a fish, an amphibian, and an invertebrate.

For purposes of the present disclosure, the term “ultra-short peptide” and “self-assembling peptide” are used interchangeably. These terms refer to a sequence containing 3-7 amino acids.

### DESCRIPTION

While the invention is susceptible to various modifications and alternative forms, specific embodiments thereof has been shown by way of example in the drawings and will be described in detail below. It should be understood, however that it is not intended to limit the invention to the particular forms disclosed, but on the contrary, the invention is to cover all modifications, equivalents, and alternatives falling within the spirit and the scope of the invention.

The development of simple strategies to make biocomposites at the nano- to micro-scale is rapidly increasing in the fields of biomedicine and catalysis for the degradation of pollutants.^{1,2} Biocomposites are usually defined as materials where biological building blocks play a crucial role in the synthesis and remain a part of materials because of certain interactions. Recently, metal, especially gold and silver, composites with peptides and proteins where they act as stabilizing and reducing agents have been developed.^{3,4} The applications of gold nanoparticles depend on their size, shape, and composition as well as their arrangement and self-organization.^{5,6} However, the preparation of gold nanoparticles with defined dimensions and controlled size and morphology and their random aggregation and compromised biocompatibility have remained to be significant challenges.⁷ To address these challenges, one strategy is to use microorganisms like bacteria and algae and those from plant extracts for the formation of gold nanoparticles, which could potentially tackle the biocompatibility concerns of gold nanoparticles.⁸ This provides a solution for problems that arise from the toxicity of reducing and stabilizing agents but the aggregation and controlled formation of gold nanoparticles remain to be a challenging task.

Other than the microbe-based synthesis of metal nanomaterials, self-assembly, a natural and spontaneous process, is a promising bottom-up approach. It depends on the noncovalent interactions (electrostatic interactions, hydrogen bonding, pi-pi stacking, and cation-pi interactions) between the components and these interactions can be controlled by various factors like the inclusion of functional groups, pH, solvents, and temperature.⁹⁻¹¹ Recently, self-assembling biomolecules, such as peptides, proteins, and oligonucleotides, have gained enormous attention in creating metallic composite nanoparticles because of their biocompatibility and phys-icochemical advantages.^{4,12-14} Therefore, self-assembling building blocks like amyloid-like peptides,¹⁵ surfactant-like peptides,¹⁶ and peptide amphiphiles¹⁷ have the potential to tune the physicochemical properties of metals and ability to control the size in a complexation process. In this strategy, peptide building blocks act not only as reducing and stabilizing agents for gold nanoparticles but also as a template for the synthesis of gold nanoparticles in a composite material. These peptide building blocks have versatile physical properties to control the aggregation of metallic nanoparticles because of their distinctive self-assembling and recognition capabilities.¹⁸

Contrary to the self-assembly approach for the formation of gold-peptide composites, generally, the metal nanoparticles have been prepared using different methods, for example, sol-gel, hydrothermal, and precipitation methods where the metal salt is mixed with some reducing agents like hydrazine, sodium citrate, sulfonic acid, and borohydrides, which could have somehow compromised the biocompatibility and could lead to severe detrimental side effects for environmental pollution.¹⁹ Other than this, often, the size and aggregation cannot be controlled because of the lack of chemical and physical interactions and the undetermined effects of reducing agents. This leads us to use the photochemical reduction methods with the help of ultrashort peptides and mild UV light. The photochemical reduction of metal ions using peptide building blocks is an interesting approach to fabricate metal-based composite nanomaterials. Ultrashort amphiphilic peptides, a class of peptides containing three to seven amino acids, can self-assemble into a well-defined nanofibrous network, mimicking the native extracellular matrix.^{20,21} Such kinds of short peptides have been used in many applications in medicine and tissue engineering, such as bioprinting,^{22,23} drug delivery,²⁴ engineered tissue models,^{25,26} and wound healing,²⁷ which reveal the biocompatibility of peptides. In our previous works, we have reported the photochemical synthesis of size-controlled biocompatible silver nanoparticles in the absence of any chemical reducing agents for antibacterial applications.^{28,29} However, to see the versatility of this photochemical synthesis of metal-peptide nanoparticle compo-sites, we used the tetramer peptide (IVFK) and gold metal salt for the reduction of small molecule pollutants. Interestingly, this ultrashort peptide not only reduces the gold salt into nanoparticles but provides a template of nanofibers for the organization of gold nanoparticles. Small molecule organic pollutants cause severe environmental and health concerns in recent days, and metal nanoparticles including gold have shown great efficiency in the degradation of different hazardous molecules.³⁰⁻³⁶ Rather than using the inorganic reagents in the synthesis of gold nanoparticles, the invention contemplates a green and biological, simple, and mechanistically understandable approach for the catalytic reduction of pollutants.

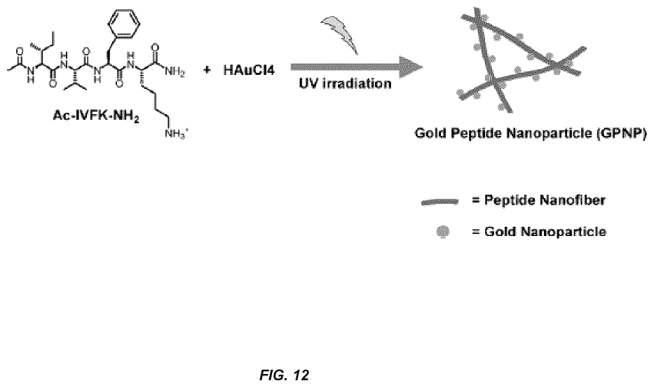

Herein, disclosed embodiments report a simple strategy using an ultrashort peptide that generates gold-peptide nanoparticle (GPNP) composites without any reducing agents through a photo-chemical reduction mechanism. The gold nanoparticles are arranged on peptide nanofibers through the multitude of noncovalent interactions and possible interactions with the amino group of the lysine amino acid in the sequence. The photoionization activity of the peptide is due to the UV light exposure of the aromatic residue, which allows the reduction of gold ions. Interestingly, the peptide acts as a reducing, capping, and stabilizing agent at the same time. The arrangements of nanoparticles over the peptide nanofibers are presented in FIG. 12, which are confirmed by different characterizations. The crystallinity of the generated gold nanoparticles was then investigated by high-resolution transmission electron micros-copy (HR-TEM) and X-ray diffraction (XRD), which demonstrate that gold nanoparticles are face-centered cubic in nature. The d-spacing was consistent in both techniques. The gold nanoparticles are well-aligned over the peptide nanofibers due to electrostatic interactions and certain binding with the amine group of the peptide, which is divulged from previous Fourier transmission infrared (FTIR) and X-ray photoelectron spectroscopy (XPS) studies. Finally, disclosed embodiments show the promising catalytic activity of gold-peptide composite nanomaterials as catalysts for the reduction of small molecule pollutants p-nitrophenol to the least toxic compound p-aminophenol in a very short time (less than 2 min) at high concentrations. The reaction rate constant for catalysis is 0.057 min⁻¹ at a 50-fold dilute sample of 2 mg/mL and 0.72 mM gold concentration in the composites. These selective bio-mineralized peptide composites via a green synthetic approach will lead to new directions for biomedical and green catalytic applications.

### Results and Discussion

### Preparation of Gold-Peptide Nanoparticle Composites

The functional groups amine and thiol as interacting sites have been reported, which can support polymers and carbon nanotubes to bind with gold nanoparticles.^{37,38} A bio-inspired tetramer oligopeptide Ac-IVFK-NH₂-based alternative pathway is proposed to fabricate colloidal composite nano-particles by triggering the biomineralization process. The peptide was designed with the amidated C-terminus and acetylated N-terminus to avoid electrostatic repulsion among the molecules in their assembled state. The short peptide sequence has hydrophobic and hydrophilic amino acids, and there are equilibria between polar and nonpolar characters, which make it water-soluble even at a very high concentration of 100 mg mL⁻¹. The natural and spontaneous process of self-assembly in peptides and proteins is ubiquitous and depends on the physical properties of building blocks to construct the supramolecular nanostructures.³⁹ These physical noncovalent interactions, such as van der Waal forces, hydrogen bonding, π-π stacking, and electrostatic forces, play a decisive and fundamental role in supramolecular chemistry for the diversification of nanomaterials.¹¹ In light of supramolecular chemistry, attractive and repulsive electrostatic forces can tune the self-assembly of short peptides on the demand of the application.⁴⁰ For example, Xing et al. reported an injectable collagen-gold hybrid hydrogel constructed through electro-static attraction for combined antitumor therapy.⁴¹ The role of hexamer oligomer peptide hydrogels in reducing the silver ions to form silver nanoparticles with the assistance of UV light was also reported.²⁸ However, the relatively small oligopeptide is not explored for the synthesis of gold-peptide nanoparticle composites, and focus was diverted to gold nanoparticles as they have been used in many applications with promising results.

Lyophilized peptide Ac-IVFK-NH₂ (2 mg/mL, 1.83 mM) was dissolved in Milli-Q water in three glass vials and mixed with HAuCl₄ solutions of concentrations of 0.18, 0.36, and 0.72 mM. All three samples were then exposed to UV light for 30 min for in situ synthesis and fabrication of the gold nanoparticles on the peptide nanofibers, without the addition of toxic reducing and capping reagents. We used the peptide in a bit low concentration because of its strong self-assembling propensity to form the self-supporting hydrogels at 4 mg/mL or higher concentrations immediately, and it was not convenient to characterize the gold nanoparticle-embedded hydrogel with traditional and commonly used techniques, such as UV-Vis spectroscopy. However, after photochemical reduction by UV exposure, the peptide hydrogel at high concentrations changed its transparent color to reddish, confirming the formation of gold nanoparticles, which are most probably trapped within the interstices of nanofibers. Inspired by the sophisticated approach of self-assembly, electrostatic complexation between the positively charged tetrapeptide motif and negatively charged [AuCl₄]-ions, which were produced under UV light, was subsequently converted into GPNP composites.

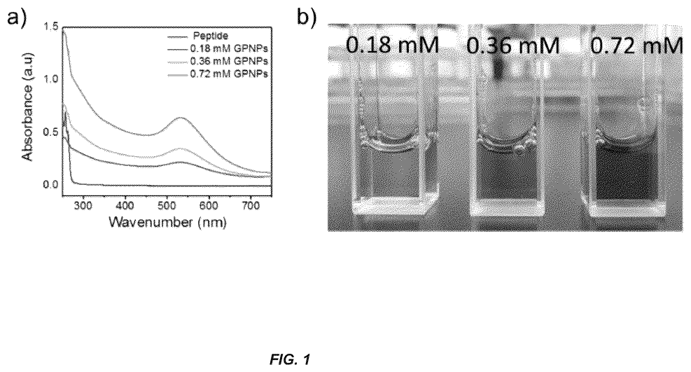

FIG. 1. illustrates UV-Vis spectra and pictures after UV light exposure at different gold concentrations, according to one embodiment of the present disclosure. FIG. 1A graphically illustrates absorption spectra of GPNP composite suspension composed of 1.83 mM IVFK peptide and gold salt at various concentrations of 0.18, 0.36, and 0.72 mM. FIG. 1B illustrates a pictorial representation of color changing of the same concentrations of GPNPs used in FIG. 1A after UV irradiation at 254 nm wavelength.

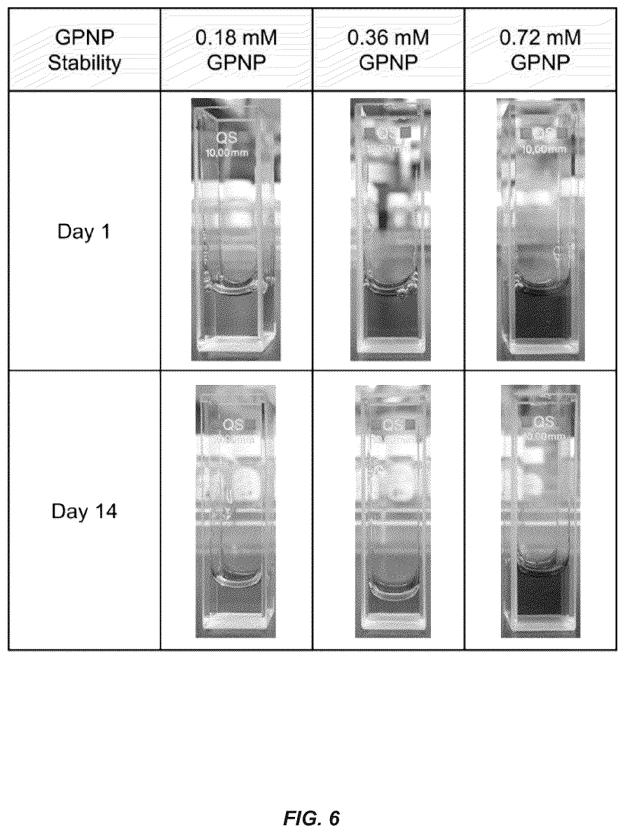

The formation of GPNP composites was confirmed by UV-Vis spectroscopy, as shown in FIG. 1A, showing the appearance of a relatively broad surface plasmonic resonance (SPR) absorption peak of gold nanoparticles at 530 nm. 42 The intensity of the absorption peak increases with increasing concentrations of the HAuCl₄ precursor, and the change in the color of the peptide and gold salt mixture from transparent to reddish also reveals the formation of gold nanoparticles with a bottom-up self-assembling approach, as illustrated in FIG. 1B. The GPNP composites were also shown to be stable for up to 14 days (FIG. 6). Furthermore, a concentration of 0.72 mM gold salt in water, as a control, was treated by UV light at 254 nm for 30 min and then analyzed by UV-Vis spectroscopy and there was no classical peak for the gold nanoparticles that appeared.

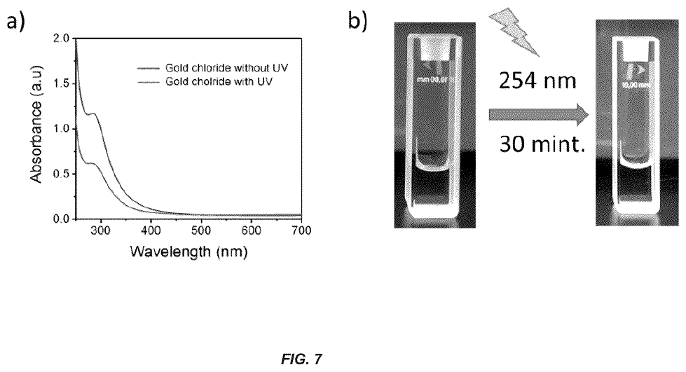

FIG. 7 illustrates photochemical reduction of HAuCl₄ in the absence of IVFK peptide, according to one embodiment of the present disclosure. No formation of gold nanoparticles was observed after 30 min of UV irradiation. The graphical representation shows the importance of peptides as reducing agents to address the absence of reducing/capping agents, as given in FIGS. 7A and 7B. FIG. 7A graphically illustrates UV-Vis absorption spectra of 0.72 mM gold chloride before and after 30 min of UV irradiation without addition of peptide. FIG. 7B shows a pictorial representation of 0.72 mM HAuCl₄ after UV irradiation in the absence of IVFK peptide. From a mechanistic point of view, here, UV light assisted the photochemical reduction process to form nanoparticles over the nanofibers of the peptide. In another study, Bent and Hay on systematically investigated the ejection of a hydrated electron (e⁻_{aq}) from the aromatic ring of a phenylalanine residue during the photoionization process.⁴³ This hydrated electron is believed to play an important role in reducing the gold ions to gold nanoparticles, and the presence of phenylalanine in IVFK critically helps the reduction process. However, the mechanism of the reduction process requires further investigation to gain greater insights from a broad perspective.

### Characterization of GPNP Composites

Generally, the metal incorporation in a biomolecular assembled material in different morphologies can be investigated by transmission electron microscopy (TEM).

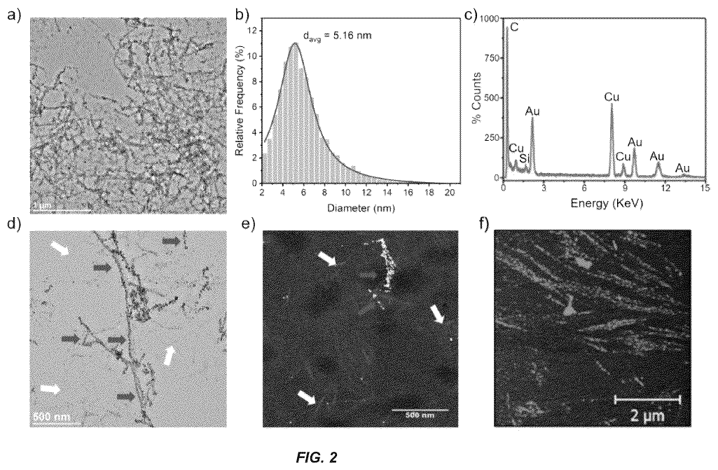

FIG. 2 illustrates a morphology characterization of GPNP composites, according to one embodiment of the present disclosure. FIG. 2A is a transmission electron microscope image indicating the formation and self-arrangement of gold nanoparticles around the nanofibers. FIG. 2B graphically illustrates TEM size distribution of GPNPs. FIG. 2C graphically illustrates an EDS spectrum confirming the presence of gold elements on the surface of a nanoparticle. FIG. 2D is a TEM image to show the nanofibers with and without gold nanoparticles. FIG. 2E represents a dark mode TEM image indicating the alignment of gold nanoparticles over the peptide nanofibers. FIG. 2F is a self-organization presented by an atomic force microscope image (AFM). Disclosed embodiments use TEM to see the formation of gold nanoparticles and self-organize over the peptide nanofibers, as demonstrated in FIG. 2A. The average size of gold nanoparticles distributed on the peptide nanofibers was approximately 5.16 nm, as given in FIG. 2B. Furthermore, energy-dispersive X-ray spectroscopy (EDS) analysis confirms the presence of a gold element in GPNP composites, as can be seen in FIG. 2C. The size distribution and arrangements of the gold nanoparticles may be due to promising biomineralization of peptides where the formation process can be in kinetic control and metal nucleation.^{44,45} In darkfield TEM, some peptide nanofibers have not shown the gold nanoparticles over the surface of nanofibers, which is attributed to the weak molecular interactions of the two components in the assembled composites. However, it also depends on the concentration of gold salt used for the synthesis of the composites and it would be possible to obtain densely populated and fully covered nanofibrous composites; we intentionally use the low concentration of components, as shown in FIGS. 2C and 2D. Additionally, disclosed embodiments use atomic force microscopy (AFM), which also identified the gold nano-particles (FIG. 2E). The size is also consistent with TEM, and most importantly, it showed the arrangement of gold nanoparticles similar to that given in the TEM images in FIG. 2D.

The amine group of the side chain of the lysine residue has previously been reported as the reduction and nucleation sites of the template-directed biomineralization reaction under acidic conditions.⁴⁴ In a similar way, we did not use any base or buffer but the pH was around 6.0 but not too acidic, which also confirms the amine role of the lysine residue for reduction along with phenylalanine. More importantly, the lysine residue helps the nucleation and arrangement of gold nanoparticles through noncovalent interactions.

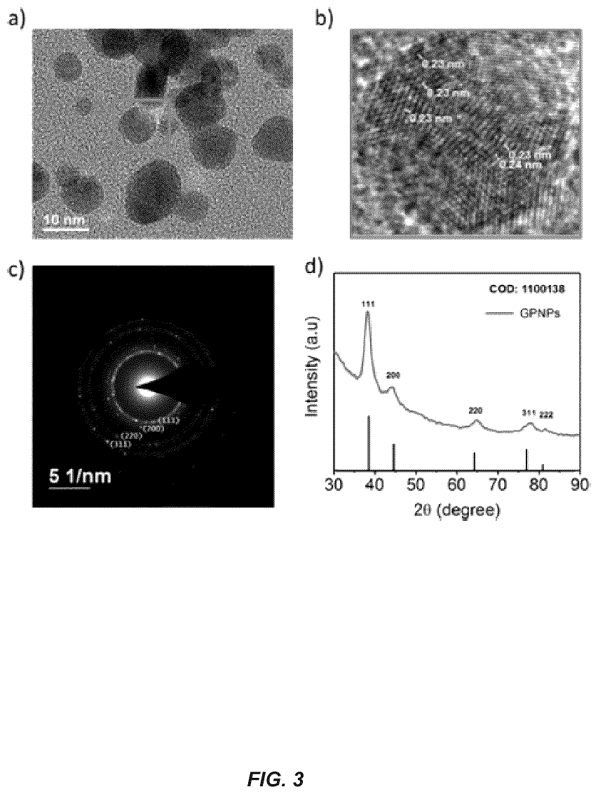

FIG. 3 illustrates a crystallinity investigation by HR-TEM and XRD, according to one embodiment of the present disclosure. FIG. 3A represents a HR-TEM image of nanoparticles. FIG. 3B is an HR-TEM lattice fringe image of GPNPs showing the (111) plane with the lattice distance 0.23 nm. FIG. 3C is a selected area electron diffraction pattern to represent the lattices. FIG. 3D graphically illustrates an XRD pattern of GPNPs. In accordance with disclosed embodiments, the crystalline nature of gold nanoparticles was investigated by selected area electron diffraction (SAED) and HR-TEM, which identified the face-centered cubic (fcc) structure with the majority of the d-spacing value of 2.3 Å from the (111) plane, as shown in FIGS. 3A and 3B. To validate the crystalline nature of gold nanoparticles in a bulk quantity of composites, X-ray powder diffraction (XRD) was used. The XRD spectrum of GPNP powder is in excellent agreement with the crystal database (COD: 1100138) of gold nanoparticles, as demon-strated in FIG. 3D.⁴⁶

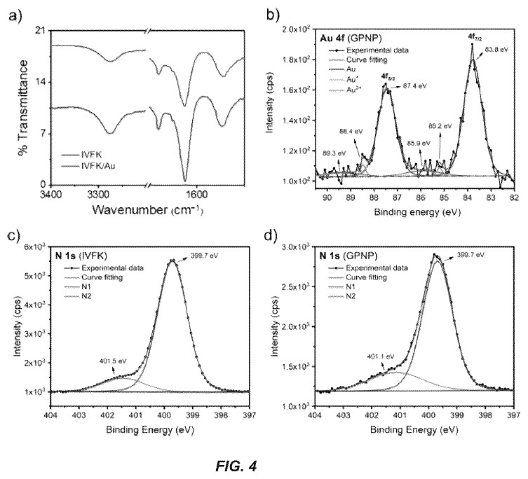

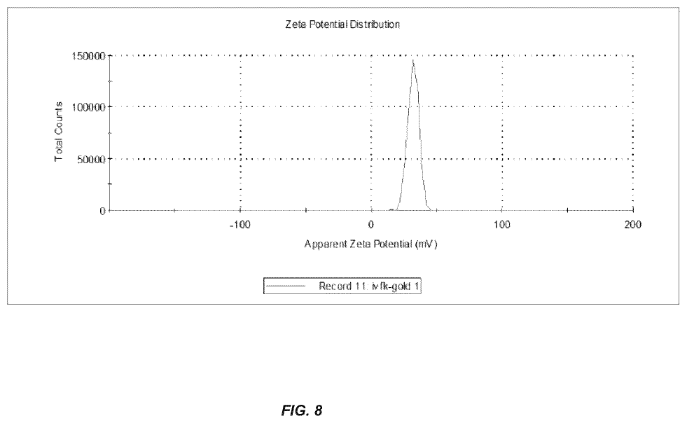

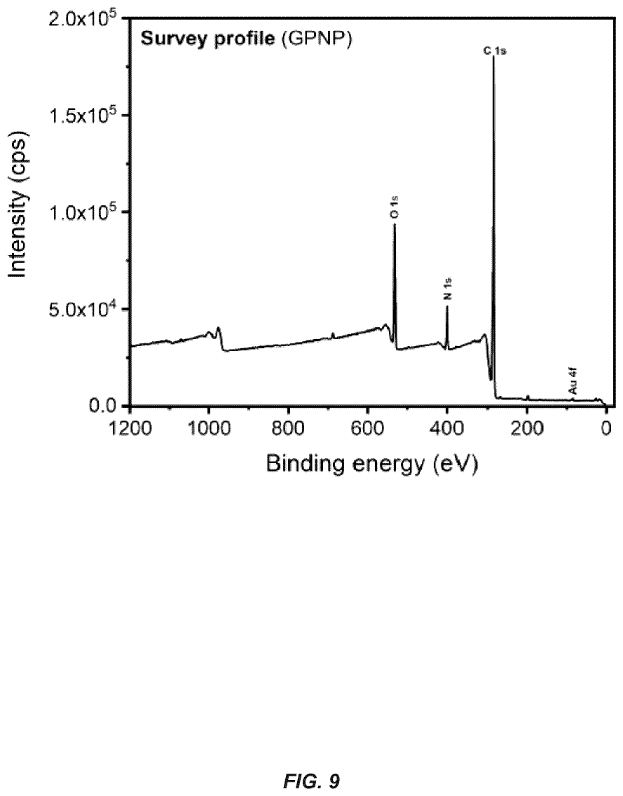

To determine the specific interaction between the peptide and gold nanoparticles to better understand the arrangement of gold nanoparticles over nanofibers, FTIR and XPS analyses were carried out. The tetramer peptide is positively charged because the lysine residue and in situ gold nanoparticles are negatively charged, as shown by the zeta potential in FIG. 8. This explains the electrostatic interaction between the two components. Furthermore, the FTIR spectra of GPNP composites confirm the interaction between the peptide template and gold after UV irradiation, as it can be seen from the blue shift of the amide A region (3275 to 3273 cm⁻¹) in —NH stretching vibration mode of the peptide. The NH bending mode (out of plane) in the amide II region (1548 to 1544 cm⁻¹) is also blue-shifted, as shown in FIG. 4A. Other characteristic peaks that remain unchanged could be due to the low concentration of gold nanoparticles being used in the sample. These shifts in NH vibrations support the hypothesis of interactions between the lysine amino acid and gold nanoparticles for their alignment on the peptide nano-templates. Furthermore, the XPS spectra were recorded to analyze the elemental composition of GPNP composites. The peaks of the survey spectrum at 531.8, 398.3, and 284.8 eV, resulting from the peptide template, confirmed the presence of oxygen, nitrogen, and carbon, respectively, as shown in FIG. 9. The high-resolution XPS spectrum of Au 4f_{7/2} shows three peaks at 83.8, 85.2, and 85.9 eV, which are attributed to the binding energies of Au(0), Au(I), and Au(III), respectively.⁴⁷ This suggests that metallic gold is dominant in GPNPs over other oxidation states. The high-resolution spectrum of N 1s of GPNPs was then compared to Ac-IVFK-NH₂ to determine the interaction between the gold and amine group of a lysine residue. Deconvolution of the N 1 s spectrum of peptide powder shows two distinct peaks of N1 at 399.7 eV and N2 at 401.5 eV, which are attributed to the amide bond 48 and protonated amine of lysine, 49 respectively, as given in FIG. 4C. In GPNP composites, the N2 peak shifted to a lower binding energy (401.1 eV), which might be due to the coordination between gold and amine groups, as shown in FIG. 4D.^{29,50} This result implies that Ac-IVFK-NH₂ can be used as both reducing and capping agents for gold nanoparticle formation through a simplistic facile strategy of photochemical reduction.

Catalytic Activity of GPNP Composites

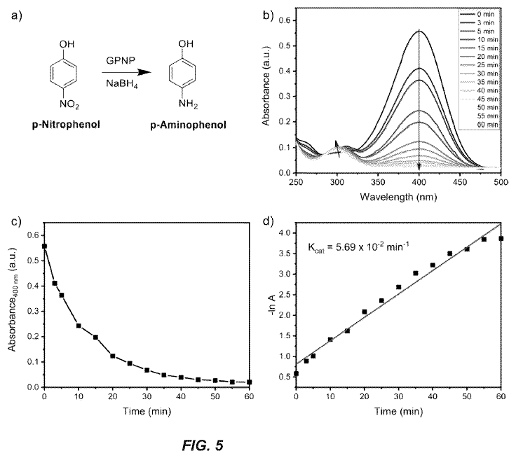

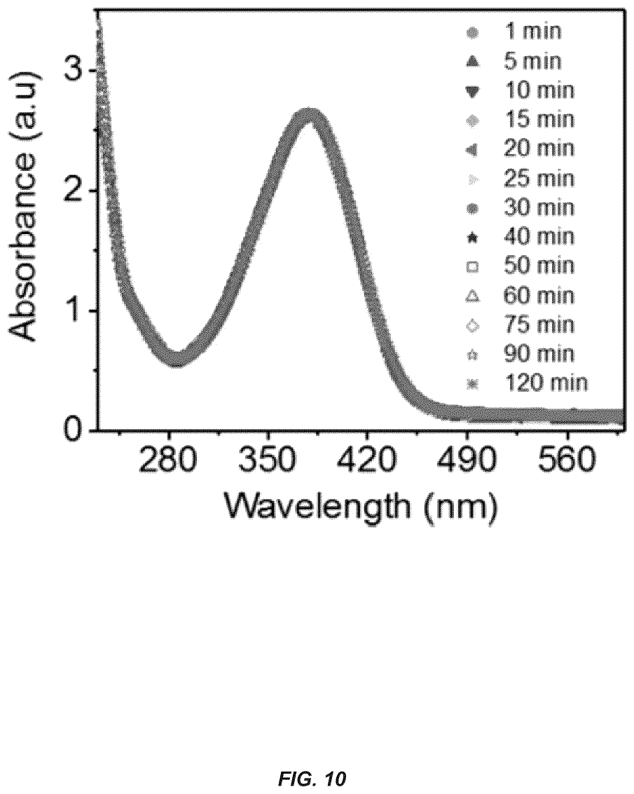

The catalytic activity of GPNP composites was investigated for the reduction of small organic molecule pollutant p-nitrophenol into p-aminophenol under ambient conditions, as given in FIG. 5A. The reaction conditions, for example, the pH and concentration, of the components of materials affect the rate of reaction, as reported by Chen and Li. Metallic nanoparticles were used to reduce p-nitrophenol in the presence of sodium borohydride (NaBH4) at a lower pH; the catalytic reduction occurred within 2 min. 51 On the first attempt, the tetrapeptide along with NaBH4 was used to reduce p-nitrophenol to p-aminophenol; however, after 2 h, the presence of p-aminophenol was not observed by UV-Vis spectropho-tometry, as shown in FIG. 10. The experiment was then repeated by introducing GPNP composites as catalysts to a mixture of p-nitrophenol and NaBH4, and the formation of p-aminophenol was detected simultaneously at around 400 nm, as given in FIG. 5B, indicating the catalytic activity of GPNP composites. Disclosed embodiments calculated the rate of reaction by using the gold-peptide composites as catalysts for the reduction of small molecule pollutant p-nitrophenol to p-aminophenol and the rate constant for catalysis is 0.057 min⁻¹ at a dilute sample of 2 mg/mL and 0.72 mM gold concentration in the composites.

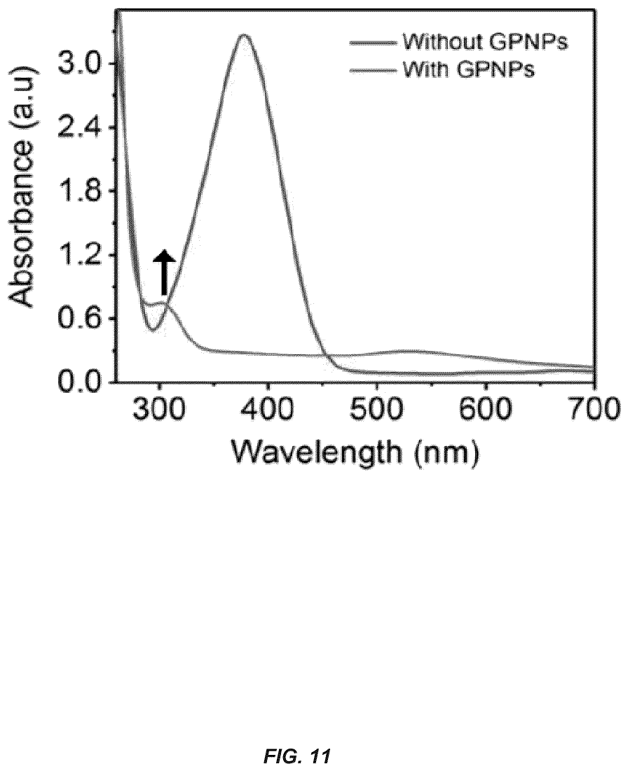

However, the rate of reaction was dependent on the concentration of peptide-gold nanocomposites because when they were used without dilution, the conversion of the organic pollutant was completed in less than 2 min, as demonstrated in FIG. 11. This catalytic reduction of p-nitrophenol to p-aminophenol is in good agreement with the previously reported literature.

### CONCLUSIONS

In summary, disclosed embodiments introduce a facile strategy to fabricate GPNP hybrids composites via self-assembly of ultrashort peptide Ac-IVFK-NH₂ and gold salt with the help of UV without any additional capping and reducing agents through a photo-chemical reduction approach. The phenylalanine and lysine amino acids in the sequence play a role in the formation of gold nanoparticles, while the lysine amino acid is mainly responsible to hold the nanoparticles on the peptide nanofibers. This attachment of gold nanoparticles is due to noncovalent interactions between two components as revealed by FTIR and XPS results. The crystallinity of nanoparticles was investigated by HR-TEM, SAED, and XRD, which demonstrate that GPNPs are face-centered cubic in nature and the d-spacing is consistent in all techniques. Furthermore, gold-peptide composites have shown a promising fast reduction of small molecule pollutant p-nitrophenol to p-aminophenol, and the reaction rate constant for catalysis is 0.057 min⁻¹ at a 50-fold dilute sample of 2 mg/mL and 0.72 mM gold concentration in the composites. However, the rate of reaction was dependent on the concentration of peptide-gold nanocomposites because when we used them without dilution, then the conversion of the organic pollutant was completed in 2 min. These peptide-metal hybrid composites via a green synthetic approach will pave the way for new approaches in biocatalysis and environmental applications.

In one embodiment, a metal-peptide nanoparticle comprising at least one metal nanoparticle; and at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3, wherein the metal-peptide nanoparticle is distributed on the peptide.

In one embodiment, the peptide comprises an amidated C-terminus and an acetylated N-terminus.

In one embodiment, the peptide is IVFK.

In one embodiment, the metal is at least one selected from the group consisting of gold and silver.

In one embodiment, the metal is gold.

In one embodiment, an average size of metal-peptide nanoparticle is 1 to 80 nm.

In one embodiment, an average size of metal-peptide nanoparticle is 1 to 20 nm.

In one embodiment, the peptide is employed in at least one of the group consisting of a medical tool kit, a fuel cell, a solar cell, an electronic cell, regenerative medicine and tissue regeneration, implantable scaffold disease model wound healing, 2D and 3D synthetic cell culture substrate, stem cell therapy, injectable therapies, biosensor development, high-throughput screening, biofunctionalized surfaces, printing biofabrication, bio-printing, and gene therapy.

In one embodiment, a biocatalyst comprising at least one metal nanoparticle; and at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3, wherein the metal nanoparticle is distributed on the peptide.

In one embodiment, a kit comprising at least one metal nanoparticle; and at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3, wherein the metal nanoparticle is distributed on the peptide.

In one embodiment, a device for applying a metal-peptide nanoparticle, wherein the metal-peptide nanoparticle comprises at least one metal nanoparticle; and at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3, wherein the metal-peptide nanoparticle is distributed on the peptide.

In one embodiment, the device is selected from the group consisting of a container with a dropper/closure device, a squeeze bottle pump spray, an airless and preservative-free spray, and an injectable device.

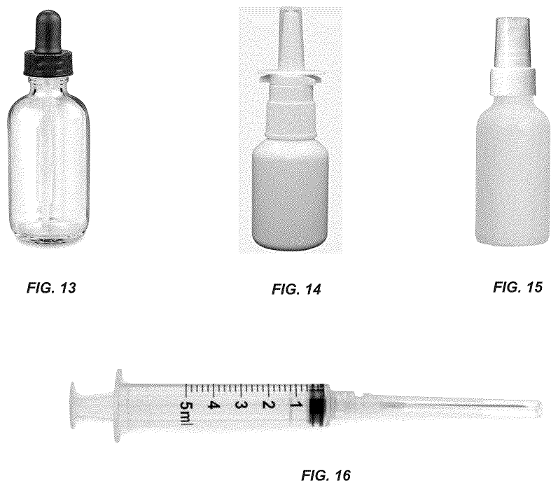

While preferred methods and devices of the present disclosure may include the device selected from the group consisting of a container with a dropper/closure device (FIG. 13), a squeeze bottle pump spray (FIG. 14), an airless and preservative-free spray (FIG. 15), and an injectable device (FIG. 16), it is readily appreciated that skilled artisans may employ other means and techniques for delivering the peptide-based adhesive material.

The injectable device (FIG. 16) may not be limited to syringe-type device. One of ordinary skill in the art would readily appreciate that any injectable device suitable for delivering the peptide-based adhesive material may be utilized according to aspects of the present disclosure.

One of ordinary skill in the art would readily appreciate that any kind of device suitable for delivering the disclosed products described in the present disclosure may be utilized.

Having described the many embodiments of the present disclosure in detail, it will be apparent that modifications and variations are possible without departing from the scope of the invention defined in the appended claims. Furthermore, it should be appreciated that all examples in the present disclosure, while illustrating many embodiments of the invention, are provided as non-limiting examples and are, therefore, not to be taken as limiting the various aspects so illustrated.

### EXAMPLES

### Materials

The tetrapeptide Ac-IVFK-NH₂ was synthesized in the laboratory using the previously reported method.^{23,52} HAuCl₄, p-nitrophenol, and sodium borohydride were purchased from Sigma Aldrich. Water of pH 6.8 with resistivity 18.2 Ω from the Milli-Q water system was used. All chemicals were used as received, unless otherwise stated here.

### Gold-Peptide Nanoparticle (GPNP) Formation.

Two milligrams of purified peptide was dissolved in Milli-Q water under vortex until complete dissolution. This peptide solution was then homogeneously mixed with 0.18, 0.36, and 0.72 mM HAuCl₄ solution. The sample mixtures were vortexed for 1 min, and the samples were exposed to UV light using a UVP CL-1000s UV Crosslinker at 254 nm wavelength with an intensity of 2.4 W/cm 2 for 30 min. The stability of the GPNP suspension was observed for up to 14 days.

### UV-Vis Spectroscopy.

The formation of gold nano-particles was characterized by ultraviolet-visible spectroscopy (Perkin Elmer UV/Vis/NIR Spectrometer Lambda 1050) using a wavelength window of 200-800 nm in 10 mm-thick quartz cuvettes.

### Transmission Electron Microscopy (TEM)

Transmission electron microscopy analysis was carried out using an FEI Titan G2 CT, fitted with a 300 kV emission gun. A 2 μL sample solution was dropped onto a carbon-coated copper grid (EMS CF300-Cu) without any additional staining reagent. The TEM grids were then dried under vacuum overnight before imaging. The SAED pattern and EDS were taken with the same instrument. The average diameter of a GPNP was measured from 5014 NPs using ImageJ and Origin software.

### Atomic Force Microscopy (AFM)

Atomic force microscope (AFM) characterization of sample morphology was carried out on a freshly cleaved mica substrate. The viscous peptide solution (5 μL) was dropcast on mica and then blotted with filter paper after 2 min. The samples were dried under a low vacuum overnight. The AFM images were taken on a Dimension Icon SPM Vecco using a tapping mode under ambient conditions. Scans were rastered using silicon-coated aluminum probes (Asylum research AC240TS-R3) with a tip radius of 9±2 nm and 70 kHz resonant frequency.

### Zeta Potential Measurements

The zeta potential of gold-peptide nanoparticle composites was measured using the Zetasizer Nano series HT Malvern at 25° C.

### Fourier Transform Infrared (FTIR)

The measurements were taken using a Thermo Scientific FTIR-ATR iS10. A background scan was measured before the sample. The spectrum was collected in a range of 500-4000 cm⁻¹, with a 1 cm⁻¹ interval. Both background and sample measurements were taken as an average over 10 scans.

### X-ray Powder Diffraction (XRD)

The crystal structure of the samples was determined using a Bruker D2 Phaser X-ray diffractometer. The lyophilized peptide-gold powders were scanned in a range of 20=10-90° with a step size of 0.02036°. The result was then compared to a gold reference with a face-centered cubic structure (COD 1100138). 46

### X-ray Photoelectron Spectroscopy (XPS)

The gold nanoparticles were lyophilized to form dry powder for XPS analysis. The XPS experiments were performed on a Kratos Axis Ultra DLD instrument equipped with a monochromatic Al Kα X-ray source (hv=1486.6 eV) operated at a power of 150 W under UHV conditions with ˜10⁻⁹ mbar. All spectra were recorded in hybrid mode using electrostatic and magnetic lenses and an aperture slot of 300 μm×700 μm. The survey and high-resolution spectra were acquired at fixed analyzer pass energies of 160 and 20 eV, respectively. The samples were mounted in floating mode to avoid differential charging. The peak fitting was performed using CasaXPS version 2.3.15 with Shirley background subtraction and the standard 70% Gaussian/30% Lorentzian line (GL30). No preliminary smoothing was conducted during analysis.

### Catalytic Performance of Gold-Peptide Nanoparticles (GPNPs)

The catalytic reduction of p-nitrophenol to p-aminophenol by GPNP composites was conducted in a solution containing 100 μL of 0.1 mM aqueous p-nitrophenol, 100 μL of 50-fold dilute GPNP composite from the initial stock concentration of 2 mg/mL peptide and 0.72 mM gold concentration, and 100 μL of 0.1 M aqueous NaBH4, which was freshly prepared under ambient conditions. As a control, the reduction of p-nitrophenol was also conducted using a high concentration of GPNPs (2 mg/mL IVFK and 0.72 mM gold concentration) with the same ratio of p-nitrophenol and NaBH4. Catalytic performance was carried out inside a UV-Vis spectroscope (Perkin Elmer UV/Vis/NIR Spectrometer Lambda 1050) to monitor the concentration change of the reactant (i.e., p-nitrophenol) and the product (i.e., p-amino-phenol).

### REFERENCES

The following references are referred to above and are incorporated herein by reference:
(1) Stephanopoulos, N.; Ortony, J. H.; Stupp, S. I. Self-Assembly for the Synthesis of Functional Biomaterials. Acta Mater. 2013, 61, 912-930.
(2) Sakiyama-Elbert, S. E.; Hubbell, J. A. Functional Biomaterials: Design of Novel Biomaterials. Ann. Rev. Mater. Res. 2001, 31, 183-201.
(3) Yu, X.; Wang, Z.; Su, Z.; Wei, G. Design, Fabrication, and Biomedical Applications of Bioinspired Peptide—Inorganic Nanoma-terial Hybrids. J. Mater. Chem. B 2017, 5, 1130-1142.
(4) Abbas, M.; Atiq, A.; Xing, R.; Yan, X. Silver-Incorporating Peptide and Protein Supramolecular Nanomaterials for Biomedical Applications. J. Mater. Chem. B 2021, 9, 4444-4458.
(5) Abdelrahman, A. I.; Mohammad, A. M.; Okajima, T.; Ohsaka, T. Fabrication and Electrochemical Application of Three-Dimensional Gold Nanoparticles: Self-assembly. J. Phys. Chem. B 2006, 110, 2798-2803.
(6) Beloglazkina, E. K.; Majouga, A. G.; Romashkina, R. B.; Zyk, N. V.; Zefirov, N. S. Gold Nanoparticles Modified with Coordination Compounds of Metals: Synthesis and Application. Rus. Chem. Rev. 2012, 81,65-90.
(7) Srinath, B. S.; Namratha, K.; Byrappa, K. Eco-Friendly Synthesis of Gold Nanoparticles by Bacillus subtilis and Their Environmental Applications. Adv. Sci. Lett. 2018, 24, 5942-5946.
(8) Khalil, A. T.; Ovais, M.; Iqbal, J.; Ali, A.; Ayaz, M.; Abbas, M.; Ahmad, I.; Devkota, H. P. Microbes-Mediated Synthesis Strategies of Metal Nanoparticles and Their Potential Role in Cancer Therapeu-tics. Semin. Cancer Biol. 2021, DOI: 10.1016/j.sem-cancer.2021.06.006.
(9) Qian, Y. X.; Wang, W. Z.; Wang, Z. H.; Jia, X. Q.; Han, Q. J.; Rostami, I.; Wang, Y. H.; Hu, Z. Y. pH-Triggered Peptide Self-Assembly for Targeting Imaging and Therapy toward Angiogenesis with Enhanced Signals. ACS Appl. Mater. Interfaces 2018, 10, 7871-7881.
(10) Zhao, Y. R.; Yang, W.; Chen, C. X.; Wang, J. Q.; Zhang, L. M.; Xu, H. Rational Design and Self-Assembly of Short Amphiphilic Peptides and Applications. Curr. Opin. Colloid Interface Sci. 2018, 35, 112-123.
(11) Zhao, X. B.; Pan, F.; Xu, H.; Yaseen, M.; Shan, H. H.; Hauser, C. A. E.; Zhang, S. G.; Lu, J. R. Molecular Self-Assembly and Applications of Designer Peptide Amphiphiles. Chem. Soc. Rev. 2010, 39, 3480-3498.
(12) Banerjee, J.; Azevedo, H. S. Crafting of Functional Biomaterials by Directed Molecular Self-Assembly of Triple Helical Peptide Building Blocks. Interface Focus 2017, 7, 20160138.
(13) Kogan, M. J.; Olmedo, I.; Hosta, L.; Guerrero, A. R.; Cruz, L. J.; Albericio, F. Peptides and metallic nanoparticles for biomedical applications. Nanomedicine 2007, 2, 287-306.
(14) Sedlak, R. H.; Hnilova, M.; Gachelet, E.; Przybyla, L.; Dranow, D.; Gonen, T.; Sarikaya, M.; Tamerler, C.; Traxler, B. An Engineered DNA-Binding Protein Self-assembles Metallic Nanostructures. Chem-BioChem 2010, 11, 2108-2112.
(15) Acar, H.; Genc, R.; Urel, M.; Erkal, T. S.; Dana, A.; Guler, M. O. Self-Assembled Peptide Nanofiber Templated One-Dimensional Gold Nanostructures Exhibiting Resistive Switching. Langmuir 2012, 28, 16347-16354.
(16) Dou, Y.; Xu, H.; Hao, J. Self-Assembly and Accurate Preparation of Au Nanoparticles in the Aqueous Solution of a Peptide A6D and a Zwitterionic C14DMAO. Soft Matter 2013, 9, 5572-5580.
(17) Hwang, L.; Chen, C.-L.; Rosi, N. L. Preparation of 1-D Nanoparticle Superstructures with Tailorable Thicknesses Using Gold-Binding Peptide Conjugates. Chem. Commun. 2011, 47, 185-187.
(18) Chen, C.-L.; Rosi, N. L. Peptide-Based Methods for the Preparation of Nanostructured Inorganic Materials. Angew. Chem., Int. Ed. 2010, 49, 1924-1942.
(19) SlepiCka, P.; SlepiCkova Kasalkova, N.; Siegel, J.; Kolska, Z.; Švorčik, V. Methods of Gold and Silver Nanoparticles Preparation. Materials 2020, 13,1.
(20) Hauser, C. A. E.; Deng, R.; Mishra, A.; Loo, Y.; Khoe, U.; Zhuang, F.; Cheong, D. W.; Accardo, A.; Sullivan, M. B.; Riekel, C.; Ying, J. Y.; Hauser, U. A. Natural Tri- to Hexapeptides Self-Assemble in Water to Amyloid (3-Type Fiber Aggregates by Unexpected α-Helical Intermediate Structures. Proc. Natl. Acad. Sci. U.S.A 2011, 108, 1361-1366.
(21) Mishra, A.; Loo, Y.; Deng, R.; Chuah, Y. J.; Hee, H. T.; Ying, J. Y.; Hauser, C. A. E. Ultrasmall Natural Peptides Self-Assemble to Strong Temperature-Resistant Helical Fibers in Scaffolds Suitable for Tissue Engineering. Nano Today 2011, 6, 232-239.
(22) Loo, Y.; Lakshmanan, A.; Ni, M.; Toh, L. L.; Wang, S.; Hauser, C. A. E. Peptide Bioink: Self-Assembling Nanofibrous Scaffolds for Three-Dimensional Organotypic Cultures. Nano Lett. 2015, 15, 6919-6925.
(23) Susapto, H. H.; Alhattab, D.; Abdelrahman, S.; Khan, Z.; Alshehri, S.; Kahin, K.; Ge, R.; Moretti, M.; Emwas, A.-H.; Hauser, C. A. E. Ultrashort Peptide Bioinks Support Automated Printing of Large-Scale Constructs Assuring Long-Term Survival of Printed Tissue Constructs. Nano Lett. 2021, 2719.
(24) Reithofer, M. R.; Chan, K.-H.; Lakshmanan, A.; Lam, D. H.; Mishra, A.; Gopalan, B.; Joshi, M.; Wang, S.; Hauser, C. A. E. Ligation of Anti-Cancer Drugs to Self-Assembling Ultrashort Peptides by Click Chemistry for Localized Therapy. Chem. Sci. 2014, 5, 625-630.
(25) Alshehri, S.; Susapto, H. H.; Hauser, C. A. E. Scaffolds from Self-Assembling Tetrapeptides Support 3D Spreading, Osteogenic Differentiation, and Angiogenesis of Mesenchymal Stem Cells. Biomacromolecules 2021, 22, 2094-2106.
(26) Ramirez-Calderon, G.; Susapto, H. H.; Hauser, C. A. E. Delivery of Endothelial Cell-Laden Microgel Elicits Angiogenesis in Self-Assembling Ultrashort Peptide Hydrogels In Vitro. ACS Appl. Mater. Interfaces 2021, 13, 29281-29292.
(27) Loo, Y.; Wong, Y.-C.; Cai, E. Z.; Ang, C.-H.; Raju, A.; Lakshmanan, A.; Koh, A. G.; Zhou, H. J.; Lim, T.-C.; Moochhala, S. M.; Hauser, C. A. E. Ultrashort Peptide Nanofibrous Hydrogels for the Acceleration of Healing of Burn Wounds. Biomaterials 2014, 35, 4805-4814.
(28) Reithofer, M. R.; Lakshmanan, A.; Ping, A. T. K.; Chin, J. M.; Hauser, C. A. E. In Situ Synthesis of Size-Controlled, Stable Silver Nanoparticles Within Ultrashort Peptide Hydrogels and Their Anti-Bacterial Properties. Biomaterials 2014, 35, 7535-7542.
(29) Seferji, K. A.; Susapto, H. H.; Khan, B. K.; Rehman, Z. U.; Abbas, M.; Emwas, A.-H.; Hauser, C. A. E. Green Synthesis of Silver-Peptide Nanoparticles Generated by the Photoionization Process for Anti-Biofilm Application. ACS Appl. Bio Mater. 2021, DOI: 10.1021/acsabm.1c01013.
(30) Bhattacharjee, A.; Ahmaruzzaman, M. CuO Nanostructures: Facile Synthesis and Applications for Enhanced Photodegradation of Organic Compounds and Reduction of p-Nitrophenol from Aqueous Phase. RSC Adv. 2016, 6, 41348-41363.
(31) Devi, T. B.; Ahmaruzzaman, M.; Begum, S. A Rapid, Facile and Green Synthesis of Ag@ AgC1Nanoparticles for the Effective Reduction of 2, 4-Dinitrophenyl Hydrazine. New J. Chem. 2016, 40, 1497-1506.
(32) Bhattacharjee, A.; Ahmaruzzaman, M. Microwave Assisted Facile and Green Route for Synthesis of CuO Nanoleaves and Their Efficacy as a Catalyst for Reduction and Degradation of Hazardous Organic Compounds. J. Photochem. Photobiol., A 2018, 353, 215-228.
(33) Bhattacharjee, A.; Ahmaruzzaman, M. A Green Approach for the Synthesis of SnO2 Nanoparticles and Its Application in the Reduction of p-Nitrophenol. Mater. Lett. 2015, 157, 260-264.
(34) Bhattacharjee, A.; Ahmaruzzaman, M.; Sinha, T. A Novel Approach for the Synthesis of SnO2 Nanoparticles and Its Application as a Catalyst in the Reduction and Photodegradation of Organic Compounds. Spectrochim. Acta, Part A 2015, 136, 751-760.
(35) Bhattacharjee, A.; Ahmaruzzaman, M. Green Synthesis of 2D CuO Nanoleaves (NLs) and Its Application for the Reduction of p-Nitrophenol. Mater. Left. 2015, 161,79-82.
(36) Sinha, T.; Ahmaruzzaman, M.; Bhattacharjee, A.; Asif, M.; Gupta, V. Lithium Dodecyl Sulphate Assisted Synthesis of Ag Nanoparticles and Its Exploitation as a Catalyst for the Removal of Toxic Dyes. J. Mol. Liq. 2015, 201, 113-123.
(37) Hussain, I.; Graham, S.; Wang, Z.; Tan, B.; Sherrington, D. C.; Rannard, S. P.; Cooper, A. I.; Brust, M. Size-Controlled Synthesis of Near-Monodisperse Gold Nanoparticles in the 1-4 nm Range Using Polymeric Stabilizers. J. Am. Chem. Soc. 2005, 127, 16398-16399.
(38) Sainsbury, T.; Ikuno, T.; Okawa, D.; Pacile, D.; Frechet, J. M. J.; Zettl, A. Self-Sssembly of Gold Nanoparticles at the Surface of Amine- and Thiol-Functionalized Boron Nitride Nanotubes. J. Phys. Chem. C 2007, 111, 12992-12999.
(39) Li, Y.; Cao, Y. The Physical Chemistry for the Self-Assembly of Peptide Hydrogels. Chin. J. Polym. Sci. 2018, 36, 366-378.
(40) Goldberger, J. E.; Berns, E. J.; Bitton, R.; Newcomb, C. J.; Stupp, S. I. Electrostatic Control of Bioactivity. Angew. Chem., Int. Ed. 2011, 50, 6292-6295.
(41) Xing, R. T.; Liu, K.; Jiao, T. F.; Zhang, N.; Ma, K.; Zhang, R. Y.; Zou, Q. L.; Ma, G. H.; Yan, X. H. Aₙ Injectable Self-Assembling Collagen-Gold Hybrid Hydrogel for Combinatorial Antitumor Photothermal/Photodynamic Therapy. Adv. Mater. 2016, 28, 3669— 3676.
(42) Haiss, W.; Thanh, N. T.; Aveyard, J.; Femig, D. G. Determination of Size and Concentration of Gold Nanoparticles from UV-Vis Spectra. Anal. Chem. 2007, 79, 4215-4221.
(43) Bent, D. V.; Hayon, E. Excited State Chemistry of Aromatic Amino Acids and Related Peptides. II. Phenylalanine. J. Am. Chem. Soc. 1975, 97, 2606-2612.
(44) Dujardin, E.; Peet, C.; Stubbs, G.; Culver, J. N.; Mann, S. Organization of Metallic Nanoparticles Using Tobacco Mosaic Virus Templates. Nano Lett. 2003, 3, 413-417.
(45) Qi, Z.; Malo de Molina, P.; Jiang, W.; Wang, Q.; Nowosinski, K.; Schulz, A.; Gradzielski, M.; Schalley, C. A. Systems Chemistry: Logic Gates Based on the Stimuli-Responsive Gel-Sol Transition of a Crown Ether-Functionalized Bis(urea) Gelator. Chem. Sci. 2012, 3, 2073-2082.
(46) Spreadborough, J.; Christian, J. W. High-Temperature X-Ray Diffractometer. J. Sci. Instrum. 1959, 36, 116-118.
(47) Yinga, F.; Wang, S.; Au, C.-T.; Lai, S.-Y. Effect of the Oxidation State of Gold on the Complete Oxidation of Isobutane on Au/CeO2 Catalysts. Gold Bull. 2010, 43, 241-251.
(48) Jansen, R. J. J.; van Bekkum, H. XPS of Nitrogen-Containing Functional Groups on Activated Carbon. Carbon 1995, 33, 1021— 1027.
(49) Zubavichus, Y.; Zharnikov, M.; Shaporenko, A.; Fuchs, 0.; Weinhardt, L.; Heske, C.; Umbach, E.; Denlinger, J. D.; Grunze, M. Soft X-ray Induced Decomposition of Phenylalanine and Tyrosine: A Comparative Study. J. Phys. Chem. A 2004, 108, 4557-4565.
(50) Das, S. K.; Dickinson, C.; Lafir, F.; Brougham, D. F.; Marsili, E. Synthesis, Characterization and Catalytic Activity of Gold Nano-particles Biosynthesized with Rhizopus Oryzae Protein Extract. Green Chem. 2012, 14, 1322-1334.
(51) Li, M. L.; Chen, G. F. Revisiting Catalytic Model Reaction p-Nitrophenol/NaBH4 using Metallic Nanoparticles Coated on Polymeric Spheres. Nanoscale 2013, 5, 11919-11927.
(52) Rauf, S.; Susapto, H. H.; Kahin, K.; Alshehri, S.; Abdelrahman, S.; Lam, J. H.; Asad, S.; Jadhav, S.; Sundaramurthi, D.; Gao, X.; Hauser, C. A. E. Self-assembling tetrameric peptides allow in situ 3D bioprinting under physiological conditions. J. Mater. Chem. B 2021, 9, 1069-1081.

All documents, patents, journal articles and other materials cited in the present application are incorporated herein by reference.

While the present disclosure has been disclosed with references to certain embodiments, numerous modification, alterations, and changes to the described embodiments are possible without departing from the sphere and scope of the present disclosure, as defined in the appended claims. Accordingly, it is intended that the present disclosure is not limited to the described embodiments, but that it has the full scope defined by the language of the following claims, and equivalents thereof.

1. A metal-peptide nanoparticle comprising:
at least one metal nanoparticle; and
at least one peptide selected from a group of peptides having a formula selected from AₙBₘX, BₘAₙX, XAₙBₘ, and XBₘAₙ,
wherein A is an aliphatic amino acid;
wherein B is comprised of at least one aromatic amino acid selected from the group consisting of: tyrosine, tryptophan, phenylalanine, and L-DOPA;
wherein X is comprised of a polar amino acid; and
wherein n being an integer being selected from 0-5 and m being an integer being selected from 0-3,
wherein the metal-peptide nanoparticle is distributed on the peptide.

2. The metal-peptide nanoparticle of claim 1, wherein the peptide comprises an amidated C-terminus and an acetylated N-terminus.

3. The metal-peptide nanoparticle of claim 1, wherein the peptide is IVFK.

4. The metal-peptide nanoparticle of claim 1, wherein the metal is at least one selected from the group consisting of gold and silver.

5. The metal-peptide nanoparticle of claim 1, wherein the metal is gold.

6. The metal-peptide nanoparticle of claim 1, wherein an average size of metal-peptide nanoparticle is 1 to 80 nm.

7. (canceled)

8. The metal-peptide nanoparticle of claim 1, wherein the peptide is employed in at least one of the group consisting of a medical tool kit, a fuel cell, a solar cell, an electronic cell, regenerative medicine and tissue regeneration, implantable scaffold disease model wound healing, 2D and 3D synthetic cell culture substrate, stem cell therapy, injectable therapies, biosensor development, high-throughput screening, biofunctionalized surfaces, printing biofabrication, bio-printing, and gene therapy.

9. (canceled)

10. (canceled)

11. (canceled)

12. (canceled)

13. (canceled)

14. (canceled)

15. (canceled)

16. A kit comprising:
at least one metal nanoparticle; and
at least one peptide selected from a group of peptides having a formula selected from AₙBₘX, BₘAₙX, XAₙBₘ, and XBₘAₙ,
wherein A is an aliphatic amino acid;
wherein B is comprised of at least one aromatic amino acid selected from the group consisting of: tyrosine, tryptophan, phenylalanine, and L-DOPA;
wherein X is comprised of a polar amino acid; and
wherein n being an integer being selected from 0-5 and m being an integer being selected from 0-3,
wherein the metal nanoparticle is distributed on the peptide.

17. The kit of claim 16, wherein the peptide comprises an amidated C-terminus and an acetylated N-terminus.

18. The kit of claim 16, wherein the peptide is IVFK.

19. The kit of claim 16, wherein the metal is at least one selected from the group consisting of gold and silver.

20. The kit of claim 16, wherein the metal is gold.

21. The kit of claim 16, wherein an average size of metal-peptide nanoparticle is 1 to 80 nm.

22. (canceled)

23. A device for applying a metal-peptide nanoparticle, wherein the metal-peptide nanoparticle comprises:
at least one metal nanoparticle; and
at least one peptide selected from a group of peptides having a formula selected from AₙBₘX, BₘAₙX, XAₙBₘ, and XBₘAₙ,
wherein A is an aliphatic amino acid;
wherein B is comprised of at least one aromatic amino acid selected from the group consisting of: tyrosine, tryptophan, phenylalanine, and L-DOPA;
wherein X is comprised of a polar amino acid; and
wherein n being an integer being selected from 0-5 and m being an integer being selected from 0-3,
wherein the metal-peptide nanoparticle is distributed on the peptide.

24. The device of claim 23, wherein the device is selected from the group consisting of a container with a dropper/closure device, a squeeze bottle pump spray, an airless and preservative-free spray, and an injectable device.

25. The device of claim 23, wherein the peptide comprises an amidated C-terminus and an acetylated N-terminus.

26. The device of claim 23, wherein the peptide is IVFK.

27. The device of claim 23, wherein the metal is at least one selected from the group consisting of gold and silver.

28. The device of claim 23, wherein the metal is gold.

29. The device of claim 23, wherein an average size of metal-peptide nanoparticle is 1 to 80 nm.

30. (canceled)
